(19) United States
(12) Patent Application Publication
(10) Pub. No.: US 2026/0260372 A1
(43) Pub. Date: Sep. 3, 2026

(54) INFORMATION PROCESSING METHOD AND INFORMATION PROCESSING APPARATUS

(71) Applicant: Fujitsu Limited, Kawasaki-shi (JP)

(72) Inventor: Fuyuka YAMADA, Kawasaki (JP)

(73) Assignee: Fujitsu Limited, Kawasaki-shi (JP)

(21) Appl. No.: 19/540,816

(22) Filed: Feb. 16, 2026

(30) Foreign Application Priority Data
Feb. 28, 2025 (JP) 2025-032353

(51) Int. Cl. G06T 7/70

(52) U.S. Cl. CPC G06T 7/70

(57) ABSTRACT

An information processing apparatus acquires first structure information indicating a first structure of a molecule and second structure information indicating a second structure estimated from a three-dimensional density map of the molecule. For each second atom included in at least a part of the second structure, the information processing apparatus calculates a first value indicating reliability of a partial structure including the second atom based on comparison between the first structure and the second structure. For each first atom of the first structure, the information processing apparatus weights a value for evaluating a position of the first atom by the first value calculated for a second atom corresponding to the evaluated first atom. Based on a second value obtained by calculation using the weighted values, the information processing apparatus determines a position and an orientation of the first structure for superposition on the three-dimensional density map.

### CROSS-REFERENCE TO RELATED APPLICATION

This application is based upon and claims the benefit of priority of the prior Japanese Patent Application No. 2025-032353, filed on Feb. 28, 2025, the entire contents of which are incorporated herein by reference.

### FIELD

The embodiments discussed herein relate to an information processing method and an information processing apparatus.

### BACKGROUND

In drug discovery and development, analyzing structures of macromolecular substances such as proteins is important. For structural analysis of molecules, a cryo-electron microscopy (cryo-EM) technique is available. When a sample is analyzed by cryo-EM, voxel data referred to as a three-dimensional density map is obtained. The three-dimensional density map alone provides only a shape of a protein, and positions and connections of atoms constituting the protein are unknown. When the positions of the atoms become known, prediction of temporal changes in the structure and prediction of binding with a ligand (a compound that is a drug candidate) become possible through simulation.

Therefore, reconstruction of a three-dimensional structure of a molecule is performed on the basis of the three-dimensional density map. The three-dimensional structure indicates positions of the atoms constituting the molecule and other information. For example, there is a technique (fitting) of obtaining a three-dimensional structure corresponding to the three-dimensional density map by deforming a known structure of the molecule so as to match the three-dimensional density map.

As an example of a technique that uses fitting, a technique relating to peptide/nucleic-acid compositions for oral/mucosal dual-mode activation of an immune defense system has been proposed. A method of imaging a protein structure using cryo-EM has also been proposed. As another example of a technique that uses cryo-EM, a method of modifying a target nucleic acid using a mutant clustered regularly interspaced short palindromic repeat (CRISPR)-Cas effector polypeptide has also been proposed. Further, as a technique that uses cryo-EM, a method for producing a synthetic single-domain monoclonal antibody library using a humanized llama nanobody framework sequence has been proposed. See, for example, the following literatures.

Japanese National Publication of International Patent Application No. 2012-519006

U.S. Patent Application Publication No. 2023/0108717

U.S. Patent Application Publication No. 2023/0407276

Japanese National Publication of International Patent Application No. 2024-535249

### SUMMARY

In one aspect, there is provided a non-transitory computer-readable recording medium storing therein a computer program that causes a computer to execute a process including: acquiring first structure information indicating a first structure of a molecule and second structure information indicating a second structure estimated based on a three-dimensional density map of the molecule; calculating, for each one of a plurality of second atoms included in at least a part of the second structure, a first value indicating reliability of a partial structure including the second atom, based on a comparison result between the first structure and the second structure; and determining a position and an orientation of the first structure for superposition on the three-dimensional density map, based on a second value, the second value being obtained by performing, for each one of a plurality of first atoms of the first structure, weighting of a value for evaluating a position of the first atom by the first value calculated for a second atom corresponding to the evaluated first atom, and calculation using the weighted values.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 illustrates an example of an information processing method according to a first embodiment;

FIG. 2 illustrates an example of a system configuration according to a second embodiment;

FIG. 3 illustrates an example of information exchanged between devices;

FIG. 4 illustrates an example of hardware of a server;

FIG. 5 illustrates an example of structural changes of a protein;

FIG. 6 illustrates an example of fitting;

FIG. 7 illustrates an example of a relationship between an initial position and ease of fitting;

FIG. 8 illustrates an example of functions included in the server for protein structure analysis;

FIG. 9 illustrates an example of structure information;

FIG. 10 illustrates an example of three-dimensional density map information;

FIG. 11 is a flowchart illustrating an example of a procedure for analyzing a three-dimensional structure;

FIG. 12 illustrates an example of reference structure generation by a main-chain tracing method;

FIG. 13 illustrates an example of reliability calculation;

FIG. 14 illustrates an example of a method for calculating root mean square deviation (RMSD);

FIG. 15 illustrates an example of rotation of an initial structure using reliability;

FIG. 16 illustrates an example of rigid-body transformation;

FIG. 17 illustrates an example of a fitting process; and

FIG. 18 illustrates an example of a relationship between an initial position and a fitting result.

### DESCRIPTION OF EMBODIMENTS

The accuracy of a three-dimensional structure obtained through fitting depends on an initial position of an existing structure during the fitting. For example, an existing structure of an object is moved to an initial position for the fitting by a rigid-body transformation that is matched to a three-dimensional density map. However, in a conventional rigid-body transformation, the existing structure is sometimes moved to an inappropriate initial position. When the initial position is inappropriate, there is a possibility that an accurate three-dimensional structure of a target molecule is not obtained even when the fitting is performed.

Hereinafter, embodiments will be described with reference to drawings. Each embodiment may be implemented in combination with another embodiment within a range not causing inconsistencies.

### (a) First Embodiment

A first embodiment is an information processing method capable of placing an initial structure of a molecule at an appropriate initial position with respect to a three-dimensional density map.

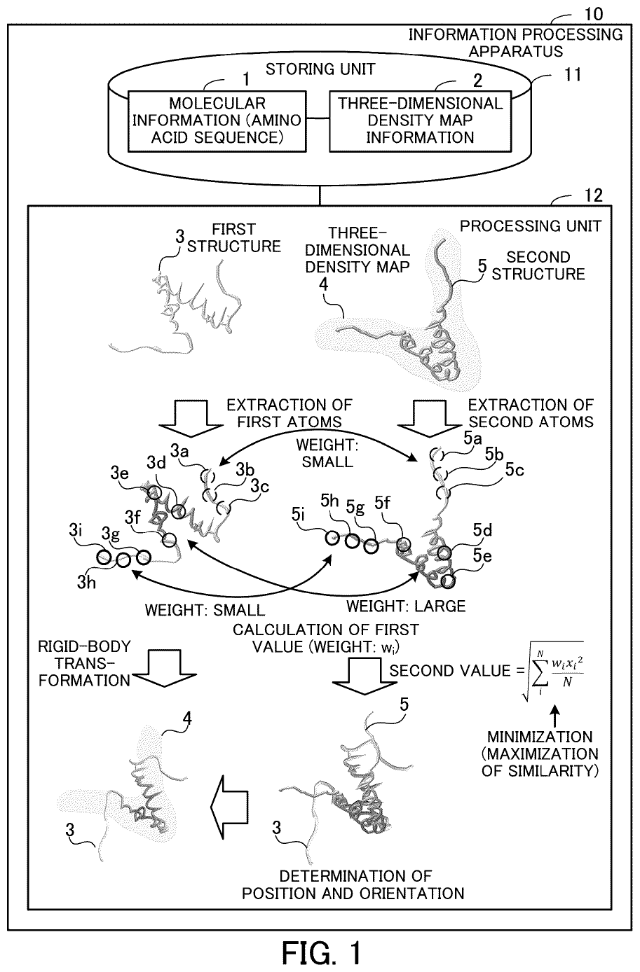

FIG. 1 illustrates an example of an information processing method according to the first embodiment. FIG. 1 illustrates an information processing apparatus 10 for executing the information processing method according to the first embodiment. The information processing apparatus 10 executes, for example, a predetermined information processing program, and thereby implements the information processing method according to the first embodiment.

The information processing apparatus 10 includes a storing unit 11 and a processing unit 12. The storing unit 11 is, for example, a memory or a storage device included in the information processing apparatus 10. The processing unit 12 is, for example, a processor included in the information processing apparatus 10. The information processing apparatus 10 may include a plurality of processors. Among a plurality of processes executed by the information processing apparatus 10, one process and another process may be executed by different processors, respectively.

The storing unit 11 stores molecular information 1 and three-dimensional density map information 2. The molecular information 1 is information indicating atoms included in a molecule that is a target of analysis and connection relationships atoms. When the molecule that is the target of analysis is a protein, information indicating, for example, an amino acid sequence is used the molecular information 1. The three-dimensional density map information 2 is information obtained by analyzing the molecule that is the target of analysis with cryo-EM. In the three-dimensional density map information 2, for each voxel obtained by dividing a three-dimensional space, a density of the molecule that is the target of analysis existing in that voxel is indicated.

The processing unit 12 analyzes a structure of the molecule that is the target of analysis on the basis of the molecular information 1 and the three-dimensional density map information 2. In doing so, the processing unit 12 superimposes a first structure 3, which is a structure of the molecule estimated from the molecular information 1, on a three-dimensional density map 4 indicated in the three-dimensional density map information 2. The processing unit 12 then deforms the first structure 3 such that the first structure 3 overlaps the three-dimensional density map 4. This deformation processing is also referred to as fitting.

An accuracy of a molecular structure obtained through the fitting depends on a position and an orientation of the first structure 3 when the first structure 3 is moved and superimposed on the three-dimensional density map 4. Therefore, the processing unit 12 determines a position and an orientation for placing the first structure 3 such that the first structure 3 is placed at an appropriate initial position with respect to the three-dimensional density map 4, by the following procedure.

The processing unit 12 acquires first structure information indicating the first structure 3 of the molecule and second structure information indicating a second structure 5 estimated on the basis of the three-dimensional density map 4 of the molecule. For example, the processing unit 12 generates the first structure information indicating the first structure 3 on the basis of the molecular information 1. The processing unit 12 also generates the second structure information indicating the second structure 5 on the basis of the three-dimensional density map information 2. The processing unit 12 may receive input of structure information indicating a known structure of the molecule that is the target of analysis and use the structure information as the first structure information.

The processing unit 12 calculates, for each of a plurality of second atoms 5a to 5i included in at least a part of the second structure 5, a first value indicating a reliability of a partial structure including the second atom, on the basis of a comparison result between the first structure 3 and the second structure 5. The partial structure including the second atom is, for example, a residue constituting a protein.

For example, the processing unit 12 sets the first value for the second atoms 5d to 5f, for which interatomic distances between atoms in the first structure 3 and atoms in the second structure 5 that are in a corresponding relationship are substantially the same, to a large value. The processing unit 12 also sets the first value for the second atoms 5a to 5c and 5g to 5i, for which interatomic distances between atoms in the first structure 3 and atoms in the second structure 5 that are in a corresponding relationship greatly differ, to a small value.

Specifically, the processing unit 12 identifies, from the second structure 5, the plurality of second atoms 5a to 5i that respectively correspond to a plurality of first atoms 3a to 3i. The processing unit 12 calculates the first value of one second atom among the plurality of second atoms 5a to 5i on the basis of (i) a positional relationship between the one second atom and surrounding atoms and (ii) a positional relationship between a first atom corresponding to the one second atom and surrounding atoms. For example, the processing unit 12 sets the first value of a second atom to be larger as distances between the second atom and surrounding atoms approximate distances between the corresponding first atom and surrounding atoms.

The processing unit 12 determines a position and an orientation of the first structure 3 for superimposing the first structure 3 on the three-dimensional density map 4 on the basis of a second value obtained by a calculation in which a value for evaluating a position of each of the plurality of first atoms 3a to 3i of the first structure 3 is weighted by the first value. The value for evaluating positions of the plurality of first atoms 3a to 3i is, for example, a distance from a corresponding second atom in the second structure 5.

The second value is, for example, information indicating a similarity between the first structure 3 and the second structure 5. The second value indicating the similarity is obtained by a calculation described below.

The processing unit 12 sets a value corresponding to a distance between an atom pair in the first structure 3 and the second structure 5 that are in a corresponding relationship (for example, a square of the distance) as a value for evaluating a position of a first atom constituting the atom pair. The processing unit 12 calculates, for each of the first atoms 3a to 3i, a value obtained by multiplying the value for evaluating the position of a first atom constituting the atom pair by a weight assigned to the first atom. The processing unit 12 sets the second value to be smaller as the multiplication results for the respective first atoms 3a to 3i are smaller. In this case, the smaller the second value is, the higher the similarity is.

When the second value indicates the similarity, the processing unit 12 determines a position and an orientation of the first structure 3 that maximize the similarity between the first structure 3 and the second structure 5 indicated by the second value. For example, when the smaller the second value is, the higher the similarity is, the processing unit 12 determines a position and an orientation of the first structure 3 that minimize the second value.

When the position and the orientation of the first structure 3 are determined, the processing unit 12 places the first structure 3 at the determined position and in the determined orientation. The placement of the first structure 3 is performed by a rigid-body transformation, and the first structure 3 is not deformed. The processing unit 12 then deforms (fits) the placed first structure 3 in accordance with the three-dimensional density map 4.

As described above, by determining the position and the orientation of the first structure 3, an appropriate position and orientation of the first structure 3 are determined. That is, values for evaluating positions of the plurality of first atoms 3a to 3i of the first structure 3 are weighted by the first value. The first value indicates a reliability of a partial structure including a second atom corresponding to an evaluated first atom, and a position of the evaluated first atom corresponding to a second atom included in a partial structure with high reliability greatly affects the second value. Therefore, by the processing unit 12 determining the position and the orientation of the first structure 3 on the basis of the second value, a partial structure of the first structure 3 corresponding to a partial structure with high reliability in the second structure 5 has its position and orientation determined such that the partial structure of the first structure 3 correctly overlaps the three-dimensional density map 4. As a result, in the fitting of the first structure 3 placed at the determined position and orientation, deformation of portions that already have high reliability is small, and accuracy of a final structure generated improves.

The processing unit 12 may move the first structure 3 tentatively placed with respect to the second structure 5 on the basis of the second value and determine a final position and orientation of the first structure 3. In this case, the first value of each of the second atoms 5a to 5i is used as a weight for a value evaluating a position of a corresponding first atom, and the processing unit 12 determines how to move the first structure 3 in accordance with the first value. For example, the processing unit 12 moves the first structure 3 such that a partial structure with high reliability overlaps the three-dimensional density map 4.

As a method of moving the first structure 3, the processing unit 12 generates, for example, a rotation matrix for rotating the first structure 3 and a translation vector for translating the first structure 3. When how to move the first structure 3 is determined, the processing unit 12 rotates and translates the first structure 3 in the determined manner and moves the first structure 3 to an initial position suitable for fitting.

The plurality of first atoms 3a to 3i of the first structure 3 is, for example, a plurality of atoms constituting a main chain in the first structure 3. By the processing unit 12 determining how to move the first structure 3 on the basis of atoms constituting the main chain, the processing unit 12 calculates an appropriate manner of movement with a small computational amount.

### Second Embodiment

A second embodiment is a computer system for reproducing a three-dimensional structure of a protein that is a target of analysis on the basis of a three-dimensional density map (also referred to as a cryo map) obtained by analyzing the protein with cryo-EM.

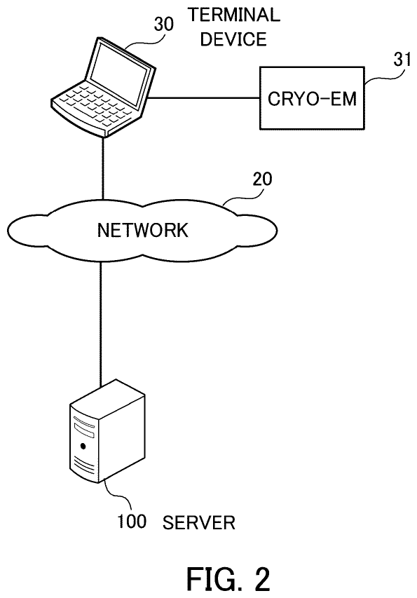

FIG. 2 illustrates an example of a system configuration according to the second embodiment. A server 100 is connected to a terminal device 30 via a network 20. The terminal device 30 is a computer used by a user who wishes to perform structural analysis of a protein. A cryo-EM 31 is connected to the terminal device 30. The cryo-EM 31 is a transmission electron microscope that analyzes a specimen at a low temperature. The server 100 is a computer capable of estimating a three-dimensional structure of a protein that is the target of analysis on the basis of a three-dimensional density map of the protein.

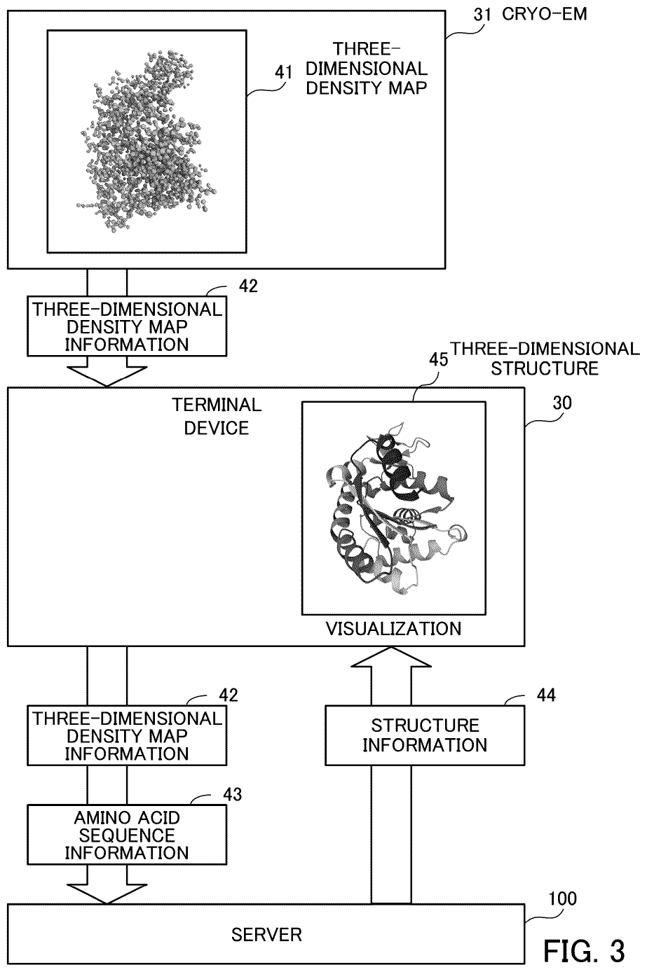

FIG. 3 illustrates an example of information exchanged between devices. When the cryo-EM 31 analyzes a protein, a three-dimensional density map 41 is generated. Three-dimensional density map information 42 indicating the three-dimensional density map 41 is then transmitted from the cryo-EM 31 to the terminal device 30. The three-dimensional density map information 42 is three-dimensional voxel data. For example, the three-dimensional density map information 42 includes X×Y×Z voxels (X, Y, and Z are natural numbers), and each voxel has a density (scalar value). The density represents a density corresponding to atoms existing in the voxel. In the three-dimensional density map 41 in FIG. 3, a voxel located deeper in a front view is represented in a darker color.

A user inputs to the terminal device 30 an instruction for structural analysis of a protein on the basis of the three-dimensional density map 41. The terminal device 30 then transmits to the server 100 a generation request for a three-dimensional structure 45 on the basis of the three-dimensional density map 41. The generation request for the three-dimensional structure 45 includes, for example, the three-dimensional density map information 42 and amino acid sequence information 43 indicating an amino acid sequence of the protein that is the target of analysis.

The server 100 generates the three-dimensional structure 45 in accordance with the generation request for the three-dimensional structure 45. Structure information 44 indicating the three-dimensional structure 45 is a set of three-dimensional coordinates (number of atoms x (x, y, z)). The server 100 transmits the structure information 44 indicating the generated three-dimensional structure 45 to the terminal device 30.

The terminal device 30 visualizes, for example, the three-dimensional structure 45 of the protein on the basis of the structure information 44. The terminal device 30 may also perform prediction of temporal changes in the structure of the protein or prediction of binding with a ligand by simulation using the structure information 44. The terminal device 30 may cause the simulation to be executed by the server 100 or another computer (not illustrated).

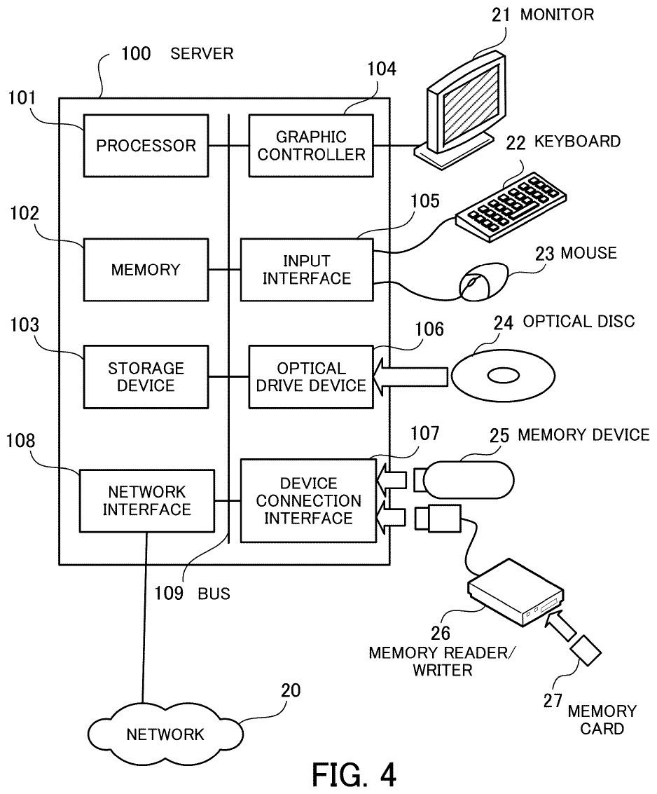

FIG. 4 illustrates an example of hardware of the server. The server 100 is controlled as a whole by a processor 101. A memory 102 and a plurality of peripheral devices are connected to the processor 101 via a bus 109.

The server 100 may be a multiprocessor system including a plurality of processors. A plurality of processors in the multiprocessor system may be collectively referred to as the processor 101. The processor 101 may also be referred to as processor circuitry. Each of the plurality of processors is capable of executing a part or all of a plurality of processes executed by the server 100. When a plurality of related processes exist, two or more processes among the plurality of processes may be executed by mutually different processors.

The processor 101 is, for example, a central processing unit (CPU), a micro processing unit (MPU), or a digital signal processor (DSP). At least a part of functions implemented by the processor 101 executing a program may be implemented by electronic circuitry such as an application specific integrated circuit (ASIC) or a programmable logic device (PLD).

The memory 102 is used as a main storage device of the server 100. At least a part of an operating system (OS) program and an application program to be executed by the processor 101 is temporarily stored in the memory 102. Various data used for processing by the processor 101 are also stored in the memory 102. As the memory 102, for example, a volatile semiconductor memory device such as random access memory (RAM) is used.

Peripheral devices connected to the bus 109 include a storage device 103, a graphic controller 104, an input interface 105, an optical drive device 106, a device connection interface 107, and a network interface 108.

The storage device 103 writes and reads data electrically or magnetically to and from a built-in recording medium. The storage device 103 is used as an auxiliary storage device of the server 100. Programs of the OS, application programs, and various data are stored in the storage device 103. As the storage device 103, for example, a hard disk drive (HDD) or a solid state drive (SSD) may be used.

The graphic controller 104 is an arithmetic device that performs image processing. The graphic controller 104 is, for example, a graphics processing unit (GPU). A monitor 21 is connected to the graphic controller 104. The graphic controller 104 displays an image on a screen of the monitor 21 in accordance with an instruction from the processor 101. As the monitor 21, a display device using organic electro luminescence (EL) or a liquid crystal display device is used. When, for example, a GPU is used as the graphic controller 104, the graphic controller 104 is also capable of executing complex numerical calculations such as matrix calculations.

A keyboard 22 and a mouse 23 are connected to the input interface 105. The input interface 105 transmits signals sent from the keyboard 22 and the mouse 23 to the processor 101. The mouse 23 is an example of a pointing device, and another pointing device may be used instead. Examples of other pointing devices include a touch panel, a tablet, a touch pad, and a trackball.

The optical drive device 106 reads data recorded on an optical disc 24 or writes data to the optical disc 24 by using laser light or the like. The optical disc 24 is a portable recording medium in which data is recorded such that the data is readable by reflection of light. Examples of the optical disc 24 include a digital versatile disc (DVD), a DVD-RAM, a compact disc read-only memory (CD-ROM), a CD-recordable (CD-R), and CD-rewritable (CD-RW).

The device connection interface 107 is a communication interface for connecting peripheral devices to the server 100. For example, a memory device 25 or a memory reader/writer 26 may be connected to the device connection interface 107. The memory device 25 is a recording medium equipped with a communication function with the device connection interface 107. The memory reader/writer 26 writes data to a memory card 27 or reads data from the memory card 27. The memory card 27 is a card-shaped recording medium.

The network interface 108 is connected to the network 20. The network interface 108 transmits and receives data to and from another computer or communication device via the network 20. The network interface 108 is a wired communication interface connected with a cable to a wired communication device such as a switch or a router. The network interface 108 may also be a wireless communication interface that is wirelessly connected to a wireless communication device such as a base station or an access point.

The server 100 implements processing functions of the second embodiment by the hardware described above. The information processing apparatus 10 described in the first embodiment may also be implemented by hardware similar to the server 100 illustrated in FIG. 4.

The server 100 implements processing functions of the second embodiment by executing a program recorded in a computer-readable recording medium. A program describing processing contents to be executed by the server 100 is recorded on various recording media. For example, a program to be executed by the server 100 is stored in the storage device 103. The processor 101 loads at least a part of a program in the storage device 103 into the memory 102 and executes the program. A program to be executed by the server 100 may also be recorded on a portable recording medium such as the optical disc 24, the memory device 25, or the memory card 27. A program stored in a portable recording medium becomes executable, for example, after being installed in the storage device 103 under control of the processor 101. The processor 101 may also directly read and execute a program from a portable recording medium.

By the server 100 having the hardware described above, a three-dimensional structure of a protein that is the target of analysis is generated on the basis of a three-dimensional density map obtained by analyzing the protein with cryo-EM.

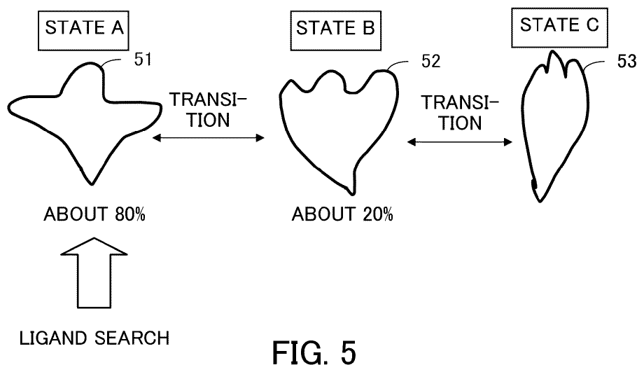

Below, with reference to FIGS. 5 to 7, importance of generating a three-dimensional structure of a protein and difficulty in doing so will be described.

FIG. 5 illustrates an example of structural changes of a protein. Atoms constituting a protein fluctuate. Therefore, a protein may take various states having different structures. In the example of FIG. 5, three states, namely state A, state B, and state C, are illustrated. A protein changes structure due to environmental changes such as temperature, pressure, or time and transitions between states.

Which state the protein is in is identified by analyzing three-dimensional structures 51 to 53 of the protein. Information regarding states of the protein under various environments is effectively utilized, for example, in drug discovery using the protein. When a protein placed in a specific environment is observed, the state A may be observed with a probability of about 80%, and the state B may be observed with a probability of about 20% (a probability of obtaining the state C is slight). When the probability of the state A is high, searching for a ligand (drug candidate) that easily binds to the state A enables efficient searching for a ligand that easily binds to the protein placed in the specific environment.

As described above, it is important to know how a three-dimensional structure of a protein that is the target of analysis transitions depending on an environment in which the protein is placed. A three-dimensional structure of a protein under a specific environment is estimated, for example, by fitting a known three-dimensional structure of the protein to a three-dimensional density map of the protein.

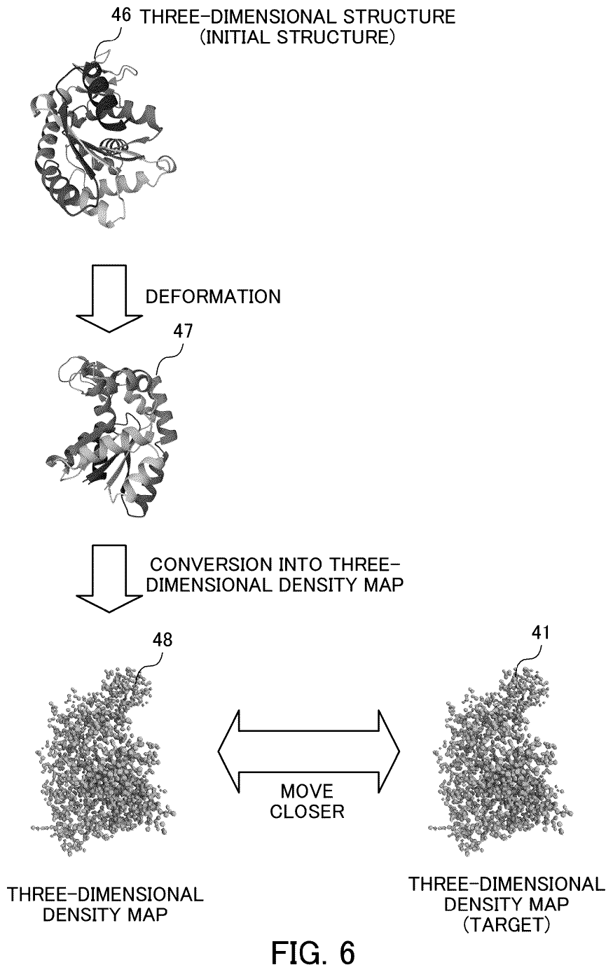

FIG. 6 illustrates an example of fitting. Three-dimensional structures 46 and 47 illustrated in FIG. 6 are visualized using data of a protein and a viewer disclosed in the paper described below.

Literature 1 (three-dimensional structure viewer Mol*): David Sehnal, Sebastian Bittrich, Mandar Deshpande, Radka Svobodová, Karel Berka, Václav Bazgier, Sameer Velankar, Stephen K Burley, Jaroslav Koča, Alexander S Rose, “Mol* Viewer: modern web app for 3D visualization and analysis of large biomolecular structures”, Nucleic Acids Research, Volume 49, Issue W1, 2 Jul. 2021, Pages W431-W437

Literature 2 (viewer-providing site RCSB PDB): Helen M. Berman, John Westbrook, Zukang Feng, Gary Gilliland, T. N. Bhat, Helge Weissig, Ilya N. Shindyalov, Philip E. Bourne, “The Protein Data Bank”, Nucleic Acids Research, Volume 28, Issue 1, 1 Jan. 2000, Pages 235-242

Literature 3 (protein 4ake): CW Müller, G J Schlauderer, J Reinstein, G E Schulz, “Adenylate kinase motions during catalysis: an energetic counterweight balancing substrate binding”, Structure, Volume 4, Issue 2, February 1996, Pages 147-156

For example, the server 100 generates the three-dimensional structure 47 representing various states of a protein that is the target of analysis by deforming the initial three-dimensional structure 46. Furthermore, the server 100 converts the deformed three-dimensional structure 47 into a three-dimensional density map 48.

The server 100 uses, as a target, a three-dimensional density map 41 obtained by analyzing a protein that is the target of analysis with cryo-EM. The server 100 repeatedly deforms the three-dimensional structure 46 such that the generated three-dimensional density map 48 becomes closer to the target three-dimensional density map 41. When a difference between the generated three-dimensional density map 48 and the target three-dimensional density map 41 becomes equal to or smaller than a predetermined value, the server 100 estimates that the three-dimensional structure 47 represents a structure of the protein that is the target of analysis.

In such fitting processing, the server 100 first moves the three-dimensional structure 46, which is an initial structure, to an initial position overlapping the target three-dimensional density map 41. In the movement, translation and rotation are performed. At this time, accuracy of the three-dimensional structure 47 after fitting depends on an initial position of the initial structure.

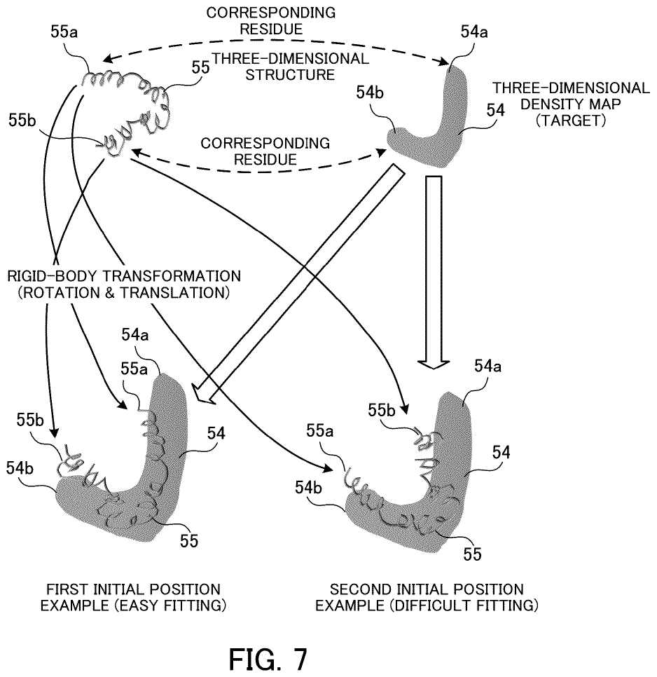

FIG. 7 illustrates an example of a relationship between an initial position and ease of fitting. The server 100 performs a rigid-body transformation (rotation and translation) of a three-dimensional structure 55 of an initial structure such that the three-dimensional structure 55 overlaps a target three-dimensional density map 54. In the example of FIG. 7, a residue at an upper end 54a of the three-dimensional density map 54 corresponds to a residue at an upper end 55a of the three-dimensional structure 55 before the rigid-body transformation. A residue at a lower end 54b of the three-dimensional density map 54 corresponds to a residue at a lower end 55b of the three-dimensional structure 55 before the rigid-body transformation.

In a first initial position example, the upper end 55a of the three-dimensional structure 55 before the rigid-body transformation overlaps the upper end 54a of the three-dimensional density map 54, and the lower end 55b of the three-dimensional structure 55 before the rigid-body transformation overlaps the lower end 54b of the three-dimensional density map 54. When the three-dimensional structure 55 is placed at such an initial position, fitting becomes easy. That is, a high-accuracy three-dimensional structure is more likely to be obtained through fitting.

On the other hand, in a second initial position example, the upper end 55a of the three-dimensional structure 55 before the rigid-body transformation overlaps the lower end 54b of the three-dimensional density map 54, and the lower end 55b of the three-dimensional structure 55 before the rigid-body transformation overlaps the upper end 54a of the three-dimensional density map 54. When the three-dimensional structure 55 is placed at such an initial position, fitting becomes difficult. That is, even if deformation of the three-dimensional structure 55 is repeated, it becomes difficult to fit the three-dimensional density map 54 with high accuracy.

As illustrated in the example of FIG. 7, when an orientation of an initial position greatly differs from an orientation of the target three-dimensional density map 54, there may be a case in which a three-dimensional structure corresponding to the three-dimensional density map 54 is not attainable even when fitting is performed. For example, when a rigid-body transformation is performed such that outer shapes are matched, and the three-dimensional density map 54 and the three-dimensional structure 55 have a common characteristic part in their outer shapes, the orientation of the three-dimensional structure 55 may be aligned with that of the three-dimensional density map 54 by using the characteristic part as a guide. On the other hand, as illustrated in FIG. 7, when the outer shapes of the three-dimensional density map 54 and the three-dimensional structure 55 greatly differ, or when multiple rigid-body transformation patterns that allow the outer shapes to overlap exist, it is difficult to align the orientations of the three-dimensional density map 54 and the three-dimensional structure 55 based only on characteristic outer shapes.

Therefore, the server 100 performs rotation of an initial structure using part of structure information corresponding to the target three-dimensional density map 54. By effectively utilizing information obtained from the target three-dimensional density map 54, appropriate rotation of the three-dimensional structure 55 becomes possible.

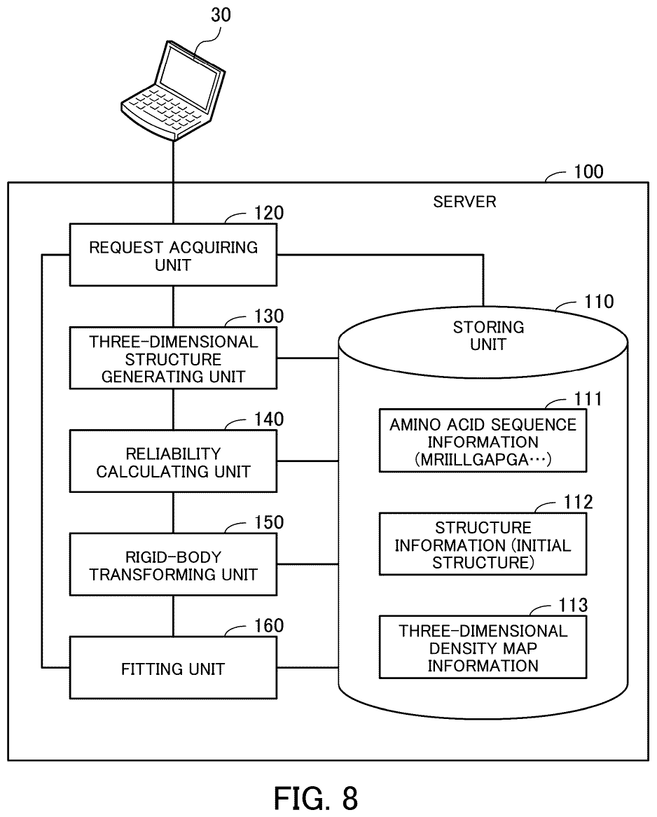

FIG. 8 illustrates an example of functions included in a server for protein structure analysis. The server 100 includes a storing unit 110, a request acquiring unit 120, a three-dimensional structure generating unit 130, a reliability calculating unit 140, a rigid-body transforming unit 150, and a fitting unit 160.

The storing unit 110 stores various types of data used for analysis of a three-dimensional structure of a protein. For example, the storing unit 110 stores amino acid sequence information 111, structure information 112, and three-dimensional density map information 113. The amino acid sequence information 111 is information indicating a sequence of amino acids constituting a protein that is a target of analysis. The amino acid sequence information 111 is, for example, a symbol string in which symbols representing amino acids are arranged. The structure information 112 is information indicating a three-dimensional structure of the protein that is the target of analysis. The three-dimensional density map information 113 is voxel data obtained by analyzing the protein that is the target of analysis with cryo-EM.

The request acquiring unit 120 receives, from the terminal device 30, an analysis request instructing analysis of a three-dimensional structure of a protein. The analysis request includes, for example, information for identifying the protein that is the target of analysis and the three-dimensional density map information 113. The analysis request may further include the amino acid sequence information 111 and the structure information 112. The request acquiring unit 120 stores information included in the analysis request in the storing unit 110 and instructs the three-dimensional structure generating unit 130 to generate a reference structure. When the request acquiring unit 120 acquires structure information indicating a generated three-dimensional structure from the fitting unit 160, the request acquiring unit 120 transmits the structure information to the terminal device 30 as an analysis result.

The three-dimensional structure generating unit 130 infers a structure of the protein on the basis of the amino acid sequence information 111. When information indicating a known structure of the protein is input, inference of the structure of the protein by the three-dimensional structure generating unit 130 is not needed.

The three-dimensional structure generating unit 130 further generates a partial structure of the three-dimensional structure of the protein that is the target of analysis on the basis of the three-dimensional density map information 113 indicating a target three-dimensional density map. Hereinafter, a partial structure generated by the three-dimensional structure generating unit 130 is referred to as a reference structure. The reference structure is, for example, information indicating positions of atoms constituting a main chain of the protein (Cα atoms) in a three-dimensional space. A Cα atom is a carbon atom closest to a carboxyl group of an amino acid.

The three-dimensional structure generating unit 130 generates the reference structure by, for example, a main-chain tracing method. The main-chain tracing method is a method of generating a partial structure by tracing a main chain of a protein. The main chain is the longest series of covalently bonded atoms. The reference structure generated by the main-chain tracing method may be an incomplete structure including missing atoms or erroneous connections. The three-dimensional structure generating unit 130 may receive manual input of atom placement from a user and generate the reference structure in accordance with the input. The three-dimensional structure generating unit 130 transmits the generated reference structure to the reliability calculating unit 140.

The reliability calculating unit 140 calculates a reliability value of each residue included in an initial structure of a three-dimensional structure that is to be generated. The reliability is, for example, a predicted local distance difference test (pLDDT) value. A pLDDT value represents reliability of a position of each residue by a value from 0 to 100. The pLDDT value is used as an index indicating a degree to which the reference structure reproduces the initial structure. A residue having a higher pLDDT value is more structurally stable and is more likely to constitute a main part of the protein. A residue having a lower pLDDT value is more likely to constitute a portion that moves flexibly. The reliability calculating unit 140 transmits the reliability of each residue to the rigid-body transforming unit 150.

The rigid-body transforming unit 150 performs rigid-body transformation of the initial structure of the three-dimensional structure on the basis of the reliability of each residue. For example, the rigid-body transforming unit 150 weights residues by pLDDT values and determines a rotation matrix and a translation vector such that a value of root mean square deviation (RMSD) becomes small.

Specifically, the rigid-body transforming unit 150 extracts Cα atoms of the same amino acid residues from each of the initial structure and the reference structure. The rigid-body transforming unit 150 superimposes the Co atoms of the initial structure on the Ca atoms of the reference structure such that an RMSD value becomes small. The rigid-body transforming unit 150 then determines a rotation matrix and a translation vector for moving the initial structure to a position at which the RMSD becomes minimum. At this time, the rigid-body transforming unit 150 calculates the RMSD weighted by pLDDT values. As a result, a rotation matrix and a translation vector suitable for highly accurate superposition of stable portions are obtained. The rigid-body transforming unit 150 rotates the initial structure of the protein in accordance with the obtained rotation matrix and translates the initial structure in accordance with the obtained translation vector.

The fitting unit 160 deforms the initial structure placed at an initial position by rigid-body transformation and fits the deformed initial structure to a three-dimensional density map obtained by cryo-EM. The fitting unit 160 transmits structure information indicating a three-dimensional structure after fitting to the request acquiring unit 120.

By the server 100 having the above-described functions, a highly accurate three-dimensional structure is generated on the basis of a three-dimensional density map of a protein. The functions of the respective elements illustrated in FIG. 8 are realized, for example, by causing the processor 101 to execute program modules corresponding to the respective elements.

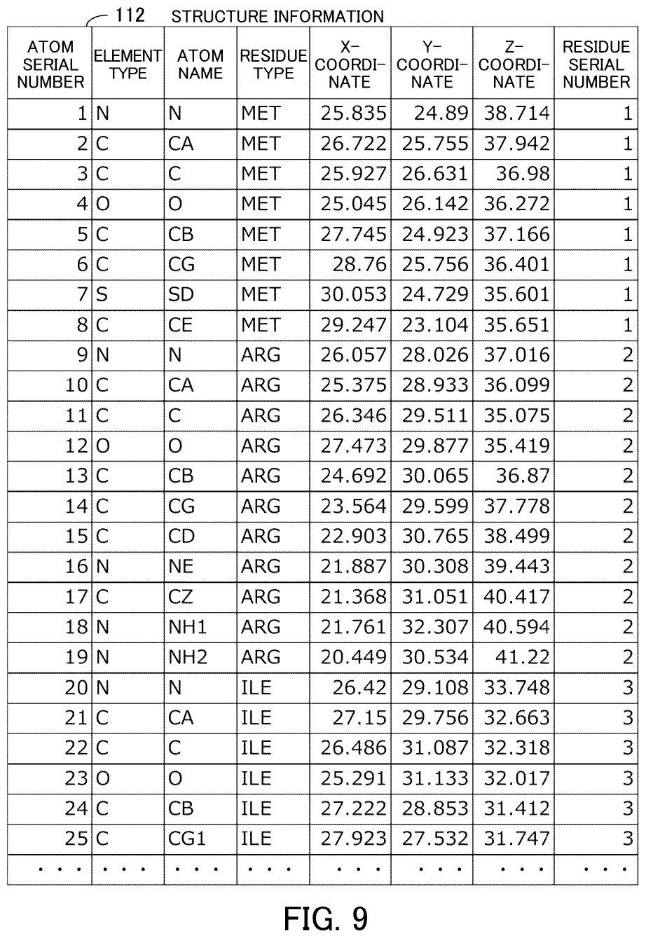

FIG. 9 illustrates an example of structure information. The structure information 112 includes, for example, records for individual atoms constituting a protein. Each record includes data such as an atom serial number, an element type, an atom name, a residue type, an x-coordinate, a y-coordinate, a z-coordinate, and a residue serial number.

The atom serial number is a number that uniquely identifies a corresponding atom. The element type indicates an element symbol of the corresponding atom. The atom name indicates a name of the corresponding atom in a protein structure. An atom described with an atom name “CA” is a Co atom. The residue type indicates a type of a residue to which the corresponding atom belongs.

The x-coordinate, the y-coordinate, and the z-coordinate are coordinates of the corresponding atom in a three-dimensional space. The residue serial number is a number that uniquely identifies a residue to which the corresponding atom belongs.

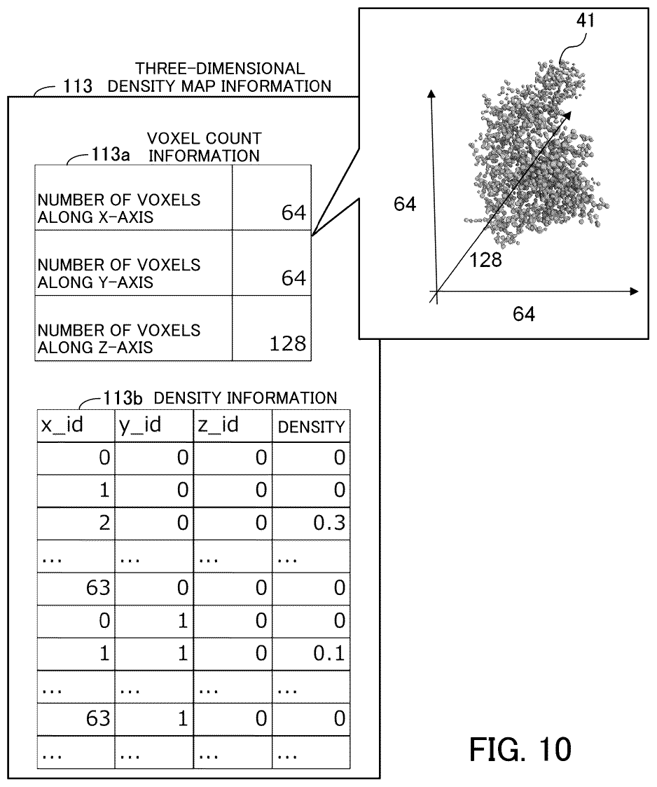

FIG. 10 illustrates an example of three-dimensional density map information. The three-dimensional density map information 113 includes, for example, voxel information 113a and density information 113b. The count voxel count information 113a indicates numbers of voxels along respective axial directions of a three-dimensional density map. A result obtained by multiplying an x-axis voxel count, a y-axis voxel count, and a z-axis voxel count represents a total number of voxels in the three-dimensional density map.

The density information 113b indicates densities of individual voxels. In the density information 113b, each voxel is uniquely identified by a numerical sequence (x_id, indicating an ordinal position of the voxel y_id, z_id) along each axial direction.

Next, a procedure for analyzing a three-dimensional structure based on a three-dimensional density map of a protein that is a target of analysis will be described.

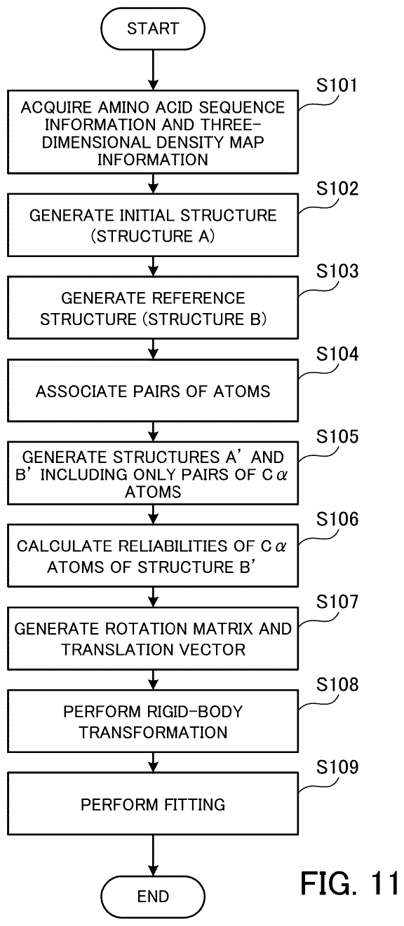

FIG. 11 is a flowchart illustrating an example of a procedure for analyzing a three-dimensional structure. The process illustrated in FIG. 11 is described below with reference to step numbers.

[Step S101] When an analysis request for a three-dimensional structure is input, the request acquiring unit 120 acquires the amino acid sequence information 111 and the three-dimensional density map information 113 from the analysis request. The request acquiring unit 120 stores the acquired amino acid sequence information 111 and three-dimensional density map information 113 in the storing unit 110. A three-dimensional density map indicated by the acquired three-dimensional density map information 113 is a target three-dimensional density map.

[Step S102] The three-dimensional structure generating unit 130 infers a protein structure (Structure A) based on the amino acid sequence information 111. The inference of the protein structure is performed, for example, using a machine learning model for predicting a protein folding structure in accordance with atomic dimensions. The three-dimensional structure generating unit 130 sets a three-dimensional structure output by the inference as an initial structure for fitting. Instead of generating a three-dimensional structure, a known three-dimensional structure may be acquired.

[Step S103] The three-dimensional structure generating unit 130 generates a reference structure (Structure B) corresponding to the target three-dimensional density map based on the three-dimensional density map information 113. For example, the three-dimensional structure generating unit 130 generates the reference structure by a main-chain tracing method.

[Step S104] The reliability calculating unit 140 associates amino acid residue pairs between Structure A and Structure B. For example, the reliability calculating unit 140 performs the association by aligning amino acid sequences. The reliability calculating unit 140 may associate pairs of atoms between Structure A and Structure B. For example, the pairs of atoms are pairs of Cα atoms.

Structure A is generated based only on the amino acid sequence information 111 and has a correct amino acid sequence (for example, “MRIILLGAPGA . . . ”). On the other hand, Structure B uses the amino acid sequence information 111, but may have an incorrect amino acid sequence (for example, “MAIILRGAPGA . . . ”) in order to match the three-dimensional density map information 113. In amino acid sequence alignment, for example, amino acid residues are compared sequentially from the beginning of the amino acid sequences of Structure A and Structure B, and identical amino acid residues are associated with each other.

[Step S105] The reliability calculating unit 140 extracts Cα atoms of each amino acid residue that forms a pair from Structure A and Structure B. The reliability calculating unit 140 generates a Structure A′ composed only of the Cα atoms extracted from Structure A. The reliability calculating unit 140 also generates a Structure B′ composed only of the Cα atoms extracted from Structure B. Since Ca atoms are part of a main chain, Structure A′ and Structure B′ represent main-chain structures.

[Step S106] The reliability calculating unit 140 calculates reliabilities of respective Cα atoms of Structure B′ based on Structure A′. The reliability is, for example, a pLDDT value.

[Step S107] The rigid-body transforming unit 150 performs a rigid-body transformation to superpose Structure A′ on Structure B′. For example, the rigid-body transforming unit 150 determines a rotation matrix and a translation vector. At this time, the rigid-body transforming unit 150 performs superposition such that a root mean square deviation (RMSD) value weighted by pLDDT values is minimized. A problem of determining a rotation matrix and a translation vector for such superposition is a nonlinear optimization problem of finding continuous variables (the rotation matrix and the translation vector) that satisfy conditions of a nonlinear function (RMSD). For example, a rotation matrix and a translation vector for rigid-body transformation are determined by a Kabsch method or an iterative closest point (ICP) method.

By determining a rotation matrix and a translation vector using only Cα atoms in this manner, main chains are reliably superposed and a computational load is reduced. A rotation matrix and a translation vector may also be determined using all atoms that form pairs. When a rotation matrix and a translation vector are determined using all atoms that form pairs, superposition that also considers side chains is achieved.

[Step S108] When the rotation matrix and the translation vector are determined, the rigid-body transforming unit 150 rotates Structure A using the rotation matrix and translates Structure A using the translation vector. Thereby, Structure A is superposed on Structure B. The position of Structure A after the movement is set as an initial position.

[Step S109] The fitting unit 160 fits Structure A at the initial position to a three-dimensional density map indicated by the three-dimensional density map information 113. That is, the fitting unit 160 deforms Structure A such that Structure A matches the three-dimensional density map as closely as possible.

In this manner, the initial structure (Structure A) is fit from an appropriate initial position, and a high-accuracy three-dimensional structure of the protein that is the target of analysis is obtained.

In the above example, a main-chain tracing method is used to generate the reference structure. In the main-chain tracing method, not all atoms constituting each amino acid indicated by the amino acid sequence information are necessarily included in the reference structure. For example, some atoms constituting an amino acid may be missing. In addition, an amino acid itself may be missing.

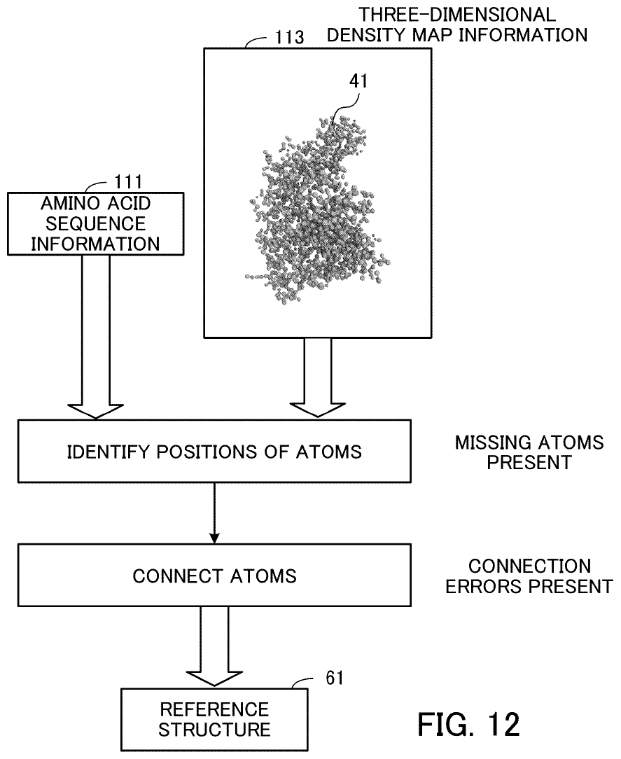

FIG. 12 illustrates an example of reference structure generation by a main-chain tracing method. For example, the three-dimensional structure generating unit 130 identifies types of atoms constituting a protein based on an amino acid sequence indicated by the amino acid sequence information 111. The three-dimensional structure generating unit 130 further determines that atoms are present in voxels having high density in the three-dimensional density map 41 indicated by the three-dimensional density map information 113. Based on this information, the three-dimensional structure generating unit 130 identifies positions of atoms whose positions are identifiable in the three-dimensional density map 41 among atoms constituting the protein. Information indicating the identified atomic positions does not include atoms whose positions are not identifiable in the three-dimensional density map 41.

The three-dimensional structure generating unit 130 connects the identified atoms based on a trajectory of a main chain. At this time, connection errors may occur. A structure obtained by connecting the identified atoms is defined as a reference structure 61.

As described above, the reference structure 61 includes missing atoms and connection errors; however, the main chain is reproduced with high accuracy. Therefore, by comparing Cα atoms constituting the main chain of the reference structure 61 with corresponding Cα atoms of an initial structure, the reliability calculating unit 140 calculates reliability regarding positional accuracy of Co atoms of the initial structure.

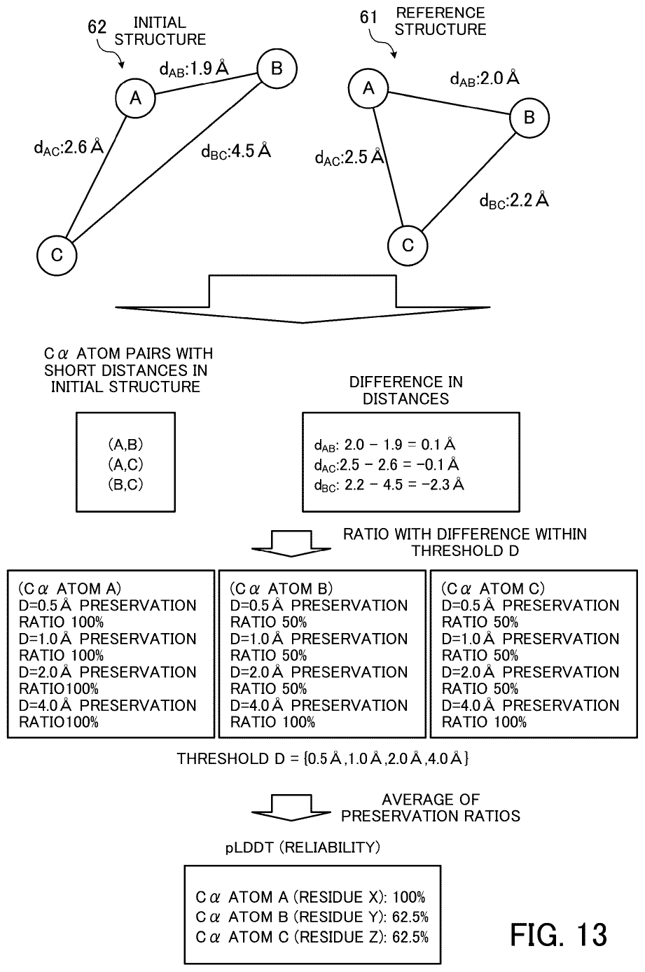

FIG. 13 illustrates an example of reliability calculation. A pLDDT value may be used as the reliability. The pLDDT value is a score obtained by calculating the local distance difference test (lDDT) using only Cα atoms. The lDDT is a score indicating how well a predicted structure reproduces a correct structure when a correct structure and a predicted structure are provided. The reliability calculating unit 140 calculates pLDDT values indicating reliability by treating an initial structure 62 as a correct structure and the reference structure 61 as a predicted structure. The calculation results indicate how well the reference structure 61 reproduces the initial structure 62.

The initial structure 62 and the reference structure 61 each include three Cα atoms “A”, “B”, and “C”. The Cα atom “A” is included in a residue “X”. The Cα atom “B” is included in a residue “Y”. The Ca atom “C” is included in a residue “Z”.

The reliability calculating unit 140 creates pairs of Cα atoms whose distances are close to each other (within a threshold R, where R is a positive real number) in the initial structure 62. In the example of FIG. 13, R is 15 Å, and three pairs {(A, B), (A, C), (B, C)} are created. The reliability calculating unit 140 also creates the same atomic pairs in the reference structure 61.

For each atomic pair, the reliability calculating unit 140 calculates a difference between a distance in the reference structure 61 and a distance in the initial structure 62. A distance difference d_{AB} of the pair (A, B) is “2.0−1.9=0.1 Å”. A distance difference d_{AC} of the pair (A, C) is “2.5-2.6=−0.1 Å”. A distance difference d_{BC} of the pair (B, C) is “2.2-4.5=−2.3 Å”.

For each atomic pair, the reliability calculating unit 140 determines whether an absolute value of the difference is within a threshold D (D is a positive real number). When the absolute value of the difference is within the threshold D, a distance of the atomic pair is determined to be preserved in the initial structure 62. For example, calculations are performed for four threshold patterns, “0.5 Å, 1 Å, 2 Å, and 4 Å”. In this case, when the threshold D is “0.5 Å, 1 Å, or 2 Å”, a distance of the pair (B, C) (difference d_{BC}=−2.3 Å) is not preserved. In other cases, distances of all pairs are preserved.

When one or both atoms of a Cα atom pair having a distance within the threshold R in the initial structure 62 do not exist in the reference structure 61, the reliability calculating unit 140 determines that a distance of the atomic pair is not preserved.

For each atom constituting an atomic pair, the reliability calculating unit 140 calculates a preservation ratio of distances to other atoms. The Cα atom “A” has a preservation ratio of 100% for all threshold D values. The Cα atom “B” has a preservation ratio of 50% when the threshold D is “0.5 Å, 1 Å, or 2 Å”, and a preservation ratio of 100% when the threshold D is “4.0 Å”. The Cα atom “C” has a preservation ratio of 50% when the threshold D is “0.5 Å, 1 Å, or 2 Å”, and a preservation ratio of 100% when the threshold D is “4.0 Å”.

The reliability calculating unit 140 sets an average value of preservation ratios for the threshold D values of each Cα atom as a final pLDDT value of the Cα atom. In the example of FIG. 13, a pLDDT value of the Cα atom “A” included in the residue “X” is “100%”. A pLDDT value of the Cα atom “B” included in the residue “Y” is “62.5%”. A pLDDT value of the Cα atom “C” included in the residue “Z” is “62.5%”.

In this manner, pLDDT values of respective Cox atoms are calculated. The pLDDT value of a Cα atom serves as reliability. Since an amino acid residue has one Cα atom, reliability is also regarded as being calculated for each residue. Reliabilities of respective Cα atoms are used as weights of RMSD when generating a rotation matrix and a translation vector.

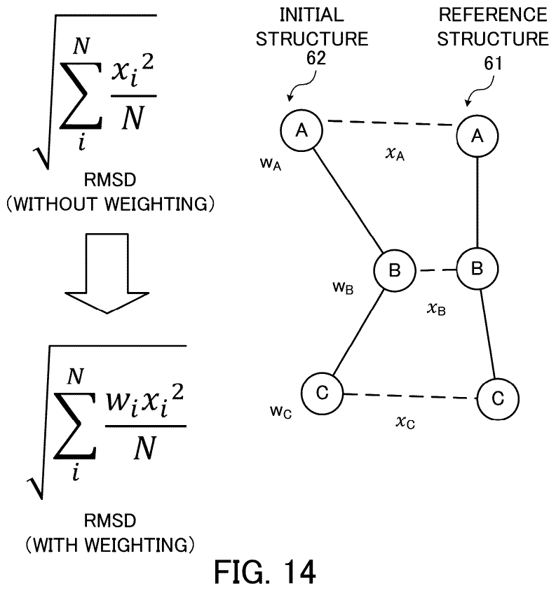

FIG. 14 illustrates an example of a method for calculating RMSD. Let a distance between a pair of corresponding Cα atoms “i” (where i is a symbol representing an atom) of the initial structure 62 and the reference structure 61 be denoted by “xi”. A distance between the Cα atom “A” of the initial structure 62 and the Cα atom “A” of the reference structure 61 is denoted by “x_{A}”. A distance between the Cα atom “B” of the initial structure 62 and the Cα atom “B” of the reference structure 61 is denoted by “x_{B}”. A distance between the Cα atom “C” of the initial structure 62 and the Cα atom “C” of the reference structure 61 is denoted by “x_{C}”. In the example of FIG. 14, the distance “x_{B}” between the Cα atoms “B” is shorter than the distances of the other Cα atoms (x_{B}≤≤x_{A}≈x_{C}).

Let a weight of a Cα atom “i” be denoted by “w₁”. A weight of the Cα atom “A” of the initial structure 62 is denoted by “w_{A}”. A weight of the Cα atom “B” of the initial structure 62 is denoted by “w_{B}”. A weight of the Cα atom “C” of the initial structure 62 is denoted by “w_{C}”.

RMSD is obtained by calculating an arithmetic mean of squares of distances of the respective Cα atoms between the reference structure 61 and the initial structure 62 and taking a square root thereof. An unweighted RMSD is calculated by the following expression.

$$\sqrt{{\sum }_{i}^{N} \frac{{x}_{i}^{2}}{N}} \qquad ( 1 )$$

N represents the number of Cα atoms included in the initial structure 62. When Expression (1) is used, minimizing RMSD results in reducing a distance of a Cα atom “A” even when a reliability of the Cα atom “A” is exceedingly low compared with those of the other Cα atoms. That is, Expression (1) is influenced by a structure having low reliability to the same extent as atoms having high reliability.

A weighted RMSD is expressed by the following expression.

$$\sqrt{{\sum }_{i}^{N} \frac{{w}_{i} {x}_{i}^{2}}{N}} \qquad ( 2 )$$

In Expression (2), a weight assigned to a Cα atom having low reliability becomes small. As a result, when RMSD is minimized, an influence of a Cα atom having low reliability is suppressed. In other words, a rotation matrix and a translation vector are obtained so that distances between pairs of Cα atoms having high reliability become small.

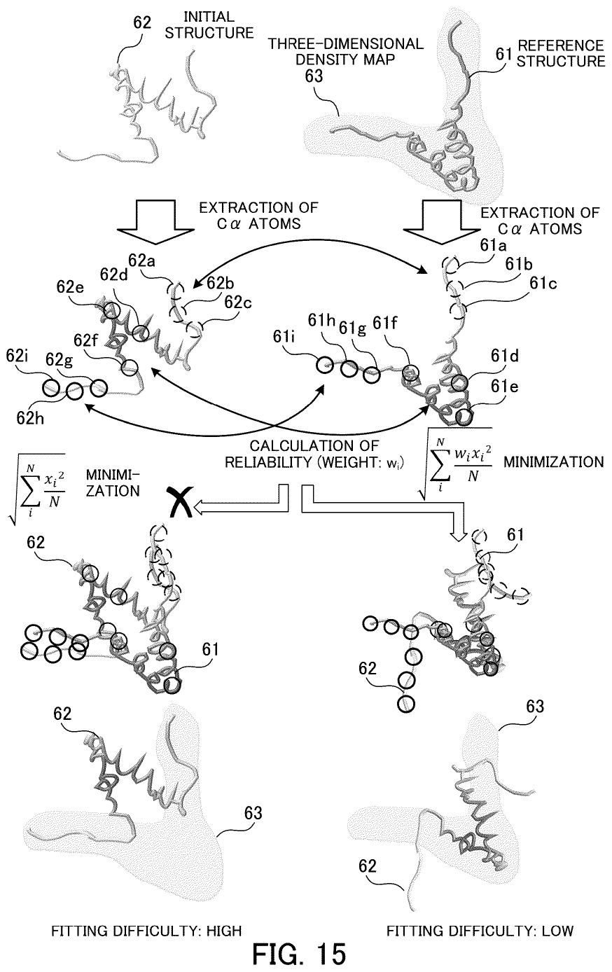

FIG. 15 illustrates an example of rotation of an initial structure using reliability. A case in which the initial structure 62 is fitted to a three-dimensional density map 63 is assumed. By extracting Cα atoms of amino acid residues from the reference structure 61 corresponding to the three-dimensional density map 63, nine Cα atoms 61a to 61i are extracted. Similarly, by extracting Cα atoms of amino acid residues from the initial structure 62, nine Cox atoms 62a to 62i are extracted.

The Cα atoms 61a to 61c of the reference structure 61 indicated by dashed-line circles respectively form pairs with the Cα atoms 62a to 62c of the initial structure 62 indicated by dashed-line circles. The Cα atoms 61d to 61f of the reference structure 61 indicated by solid-line circles respectively form pairs with the Cα atoms 62d to 62f of the initial structure 62 indicated by solid-line circles. The Cα atoms 61g to 61i of the reference structure 61 indicated by double-line circles respectively form pairs with the Cα atoms 62g to 62i of the initial structure 62 indicated by double-line circles.

Here, reliabilities of the respective Cα atoms 61a to 61i of the reference structure 61 are calculated. At this time, reliabilities of the Cα atoms 61d to 61f are assumed to be larger in value (that is, to be higher) than those of the other Cα atoms 61a to 61c and 61g to 61i. That is, a region including the Cα atoms 61d to 61f maintains inter-Cα-atomic distances at substantially the same level as those of the initial structure 62.

The lower left part of FIG. 15 illustrates an example in which the initial structure 62 is superimposed on the reference structure 61 so that an unweighted RMSD (see Expression (1)) becomes small. In this case, the initial structure 62 is oriented so as to be pulled by a region including the Cα atoms 62a to 62c and a region including the Cα atoms 62g to 62i. When an unweighted RMSD is used, a central helix region (a region including the Cα atoms 62d to 62f) is largely displaced from the three-dimensional density map 63, which makes subsequent fitting difficult. The lower right part of FIG. 15 illustrates an example in which the initial structure 62 is superimposed on the reference structure 61 so that a weighted RMSD (see Expression (2)) becomes small. In this case, regions including the Cα atoms 62a to 62c and 62g to 62i having low reliability are easily moved by rotation of the initial structure 62. That is, even when the Cα atoms 62a to 62c and 62g to 62i of the initial structure 62 move away from the Cα atoms 61a to 61c and 61g to 61i of the reference structure 61 due to rotation of the initial structure 62, an influence on RMSD is small. On the other hand, RMSD is reduced by bringing a region including the Cα atoms 62d to 62f having high reliability closer to the Cα atoms 61d to 61f of the reference structure 61.

Accordingly, superimposition focusing particularly on Cα atoms having high reliability is performed, and an important secondary structure region of a protein in the initial structure 62 is superimposed on the three-dimensional density map 63. In this case, in subsequent fitting, for example, atoms having low reliability and being easily movable due to temporal changes are preferentially moved. By lowering a priority of moving atoms having high reliability, collapse of interatomic distance relationships due to forcibly moving atoms having high reliability in fitting is suppressed. As a result, loss of natural protein-like characteristics is suppressed. Moreover, since fitting is performed while maintaining a shape of a region of the initial structure having high reliability, a difficulty of fitting is reduced.

When a rotation matrix and a translation vector that reduce a weighted RMSD are obtained, rigid-body transformation is performed based on the rotation matrix and the translation vector.

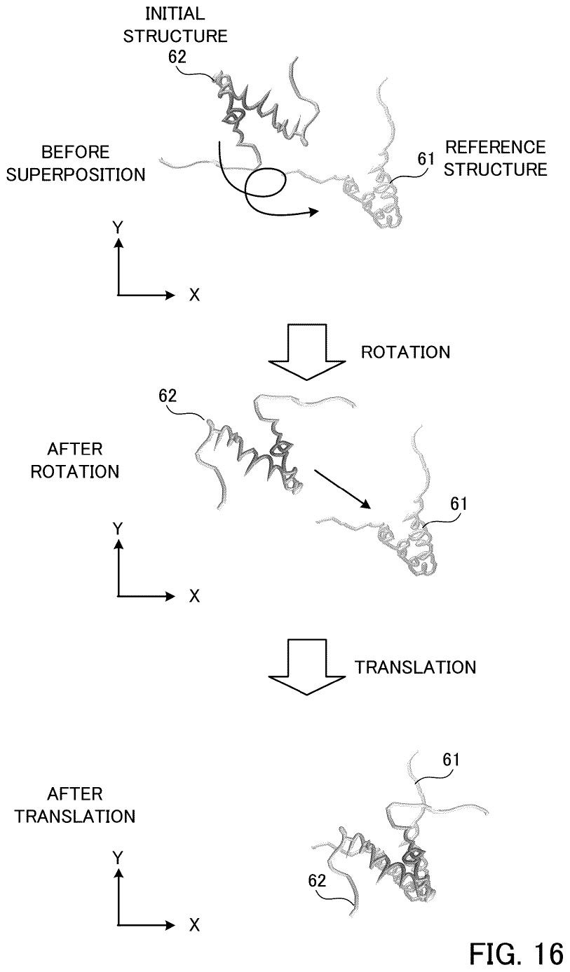

FIG. 16 illustrates an example of rigid-body transformation. A generated rotation matrix is a 3×3 matrix. A generated translation vector is a 1×3 vector. The rigid-body transforming unit 150 rotates the initial structure 62 in a three-dimensional space by using the rotation matrix. The rigid-body transforming unit 150 then translates the rotated initial structure 62 by using the translation vector. As a result, the initial structure 62 is moved to an initial position suitable for fitting.

Based on the initial structure that has been moved to the initial position, the fitting unit 160 performs fitting.

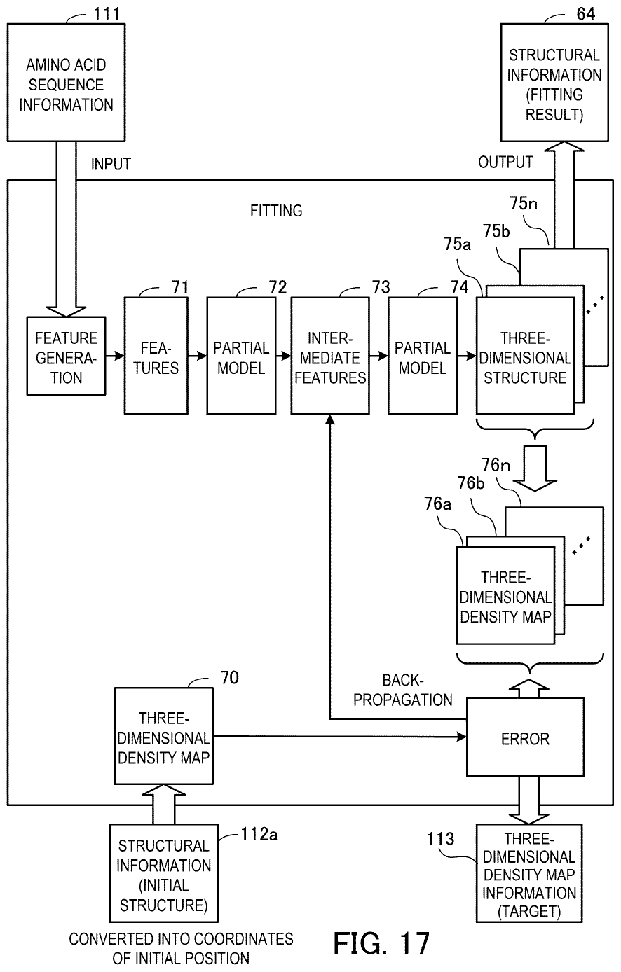

FIG. 17 illustrates an example of a fitting process. The fitting is performed, for example, using fitting fold to map (FFM). FFM is a technique for fitting a three-dimensional structure of an initial structure to a target three-dimensional density map by moving the three-dimensional structure using an artificial intelligence (AI) model. The AI model of FFM is divided, for example, into a plurality of partial models 72 and 74.

The fitting unit 160 generates features 71 based on the amino acid sequence information 111. The fitting unit 160 inputs the generated features 71 to the first partial model 72, performs calculation in accordance with the partial model 72, and obtains intermediate features 73 as output.

The fitting unit 160 also generates a three-dimensional density map 70 at step zero based on structural information 112a of the initial structure, in which positions of respective atoms have been converted into coordinates of the position initial by rigid-body transformation. The fitting unit 160 calculates an error between the generated three-dimensional density map 70 and the target three-dimensional density map indicated by the three-dimensional density map information 113. The fitting unit 160 then updates the intermediate features 73 as error backpropagation processing. For example, the fitting unit 160 calculates how a loss function changes when the intermediate features 73 are slightly changed, and updates the intermediate features 73 so as to minimize the error based on the calculated change.

The fitting unit 160 inputs the intermediate features 73 updated by backpropagation to the second partial model 74, performs calculation in accordance with the partial model 74, and obtains a three-dimensional structure 75a at step one as output.

The fitting unit 160 generates a three-dimensional density map 76a based on the three-dimensional structure 75a and calculates a difference between the generated three-dimensional density map 76a and the target three-dimensional density map indicated by the three-dimensional density map information 113. Thereafter, the fitting unit 160 repeats updating of the intermediate features 73 by error backpropagation, generation of a three-dimensional structure by the partial model 74, generation of a three-dimensional density map, and calculation of an error between the generated three-dimensional density map 76a and the target three-dimensional density map. As a result, three-dimensional structures 75b, . . . , 75n and three-dimensional density maps 76b, . . . , 76n at step two and subsequent steps are generated.

Such iterative processing is continued until a termination condition is satisfied. The termination condition is, for example, that an error becomes equal to or smaller than a predetermined value. When the three-dimensional density map 76n at step n (where n is an integer of zero or more) satisfies the termination condition, the fitting unit 160 outputs structural information 64 indicating the three-dimensional structure 75n at step n as a fitting result.

In this manner, a three-dimensional structure that fits the target three-dimensional density map is obtained. By updating the intermediate features 73 through error backpropagation, knowledge reflected in a large number of parameters possessed by the AI model of FFM is utilized to a maximum extent. That is, destruction of existing parameters (forgetting of knowledge) caused by retraining of the AT model is avoided, and generation of a highly reliable three-dimensional structure is achieved.

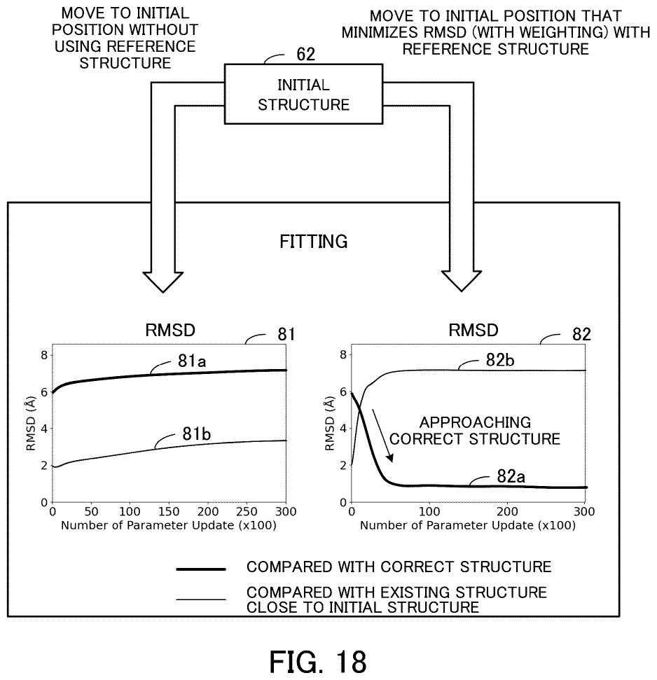

FIG. 18 illustrates an example of a relationship between an initial position and a fitting result. FIG. 18 depicts results obtained by estimating a three-dimensional structure from a three-dimensional density map of a protein for which a correct structure is known.

A graph 81 represents an evaluation result of a three-dimensional structure generated by fitting when the initial structure 62 is moved to an initial position without using a reference structure. The horizontal axis of the graph 81 represents the number of updates of intermediate features in fitting. The vertical axis of the graph 81 represents RMSD of a three-dimensional structure generated using updated intermediate features. A thick line 81a indicates changes in RMSD obtained when the generated three-dimensional structure is compared with the reference structure. A thin line 81b indicates changes in RMSD obtained when the generated three-dimensional structure is compared with an existing structure close to the initial structure 62.

A graph 82 represents an evaluation result of a three-dimensional structure generated by fitting when a position that minimizes RMSD with a reference structure (including weighting according to reliability) is used as an initial position of the initial structure 62. The horizontal axis of the graph 82 represents the number of updates of intermediate features in fitting. The vertical axis of the graph 82 represents RMSD of a three-dimensional structure generated using updated intermediate features. A thick line 82a indicates changes in RMSD obtained when the generated three-dimensional structure is compared with the reference structure. A thin line 82b indicates changes in RMSD obtained when the generated three-dimensional structure is compared with an existing structure close to the initial structure 62.

In the example of the graph 81, even when updating of intermediate features is repeated in fitting, the generated three-dimensional structure does not approach the correct structure. This is because an initial position of the initial structure 62 is inappropriate. On the other hand, in the example of the graph 82, by repeating updating of intermediate features in fitting, the generated three-dimensional structure rapidly approaches the correct structure. This is because the initial structure 62 is arranged at an appropriate initial position.

In this manner, by moving the initial structure 62 to a position and orientation that minimize weighted RMSD with a reference structure and performing fitting, a highly accurate three-dimensional structure is generated.

### (c) Other Embodiments

In the second embodiment, fitting is performed using FFM; however, fitting using a technique other than FFM is also applicable. For example, fitting using molecular dynamics flexible fitting (MDFF) is applicable. MDFF is a method in which an initial structure (a structure in a state different from a target structure of the same protein) is brought closer to a target three-dimensional density map by molecular dynamics simulation. The target structure is a structure corresponding to the three-dimensional density map. In MDFF, when calculating a force acting on particles, a difference between a three-dimensional density map of a current structure and a three-dimensional density map of a target structure is calculated, and a force that moves the structure closer to the density map is added based on the difference.

According to one aspect, an initial structure of a molecule is arranged at an appropriate initial position with respect to a three-dimensional density map.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable recording medium storing therein a computer program that causes a computer to execute a process comprising:
acquiring first structure information indicating a first structure of a molecule and second structure information indicating a second structure estimated based on a three-dimensional density map of the molecule;
calculating, for each one of a plurality of second atoms included in at least a part of the second structure, a first value indicating reliability of a partial structure including the second atom, based on a comparison result between the first structure and the second structure; and
determining a position and an orientation of the first structure for superposition on the three-dimensional density map, based on a second value, the second value being obtained by performing, for each one of a plurality of first atoms of the first structure, weighting of a value for evaluating a position of the first atom by the first value calculated for a second atom corresponding to the evaluated first atom, and calculation using the weighted values.

2. The non-transitory computer-readable recording medium according to claim 1, wherein the process further includes placing the first structure at the determined position in the determined orientation.

3. The non-transitory computer-readable recording medium according to claim 2, wherein the process further includes deforming the placed first structure in accordance with the three-dimensional density map.

4. The non-transitory computer-readable recording medium according to claim 1, wherein the calculating of the first value includes calculating the first value of the second atom based on a positional relationship between the second atom and atoms surrounding the second atom in the second structure and a positional relationship between a first atom corresponding to the second atom and atoms surrounding the first atom among the plurality of first atoms of the first structure.

5. The non-transitory computer-readable recording medium according to claim 1, wherein the calculating of the first value includes using, as the plurality of second atoms, a plurality of atoms constituting a main chain in the second structure.

6. The non-transitory computer-readable recording medium according to claim 1, wherein the determining of the position and the orientation of the first structure includes generating, based on the second value, a rotation matrix for rotating the first structure and a translation vector for translating the first structure.

7. The non-transitory computer-readable recording medium according to claim 1, wherein the determining of the position and the orientation of the first structure includes calculating the second value indicating similarity between the first structure and the second structure.

8. The non-transitory computer-readable recording medium according to claim 7, wherein the determining of the position and the orientation of the first structure includes determining the position and the orientation of the first structure that maximize the similarity indicated by the second value.

9. An information processing method comprising:
acquiring, by a processor, first structure information indicating a first structure of a molecule and second structure information indicating a second structure estimated based on a three-dimensional density map of the molecule;
calculating, by the processor, for each one of a plurality of second atoms included in at least a part of the second structure, a first value indicating reliability of a partial structure including the second atom, based on a comparison result between the first structure and the second structure; and
determining, by the processor, a position and an orientation of the first structure for superposition on the three-dimensional density map, based on a second value, the second value being obtained by performing, for each one of a plurality of first atoms of the first structure, weighting of a value for evaluating a position of the first atom by the first value calculated for a second atom corresponding to the evaluated first atom, and calculation using the weighted values.

10. An information processing apparatus comprising:
a memory; and
a processor coupled to the memory and the processor configured to:
acquire first structure information indicating a first structure of a molecule and second structure information indicating a second structure estimated based on a three-dimensional density map of the molecule,
calculate, for each one of a plurality of second atoms included in at least a part of the second structure, a first value indicating reliability of a partial structure including the second atom, based on a comparison result between the first structure and the second structure, and
determine a position and an orientation of the first structure for superposition on the three-dimensional density map, based on a second value, the second value being obtained by performing, for each one of a plurality of first atoms of the first structure, weighting of a value for evaluating a position of the first atom by the first value calculated for a second atom corresponding to the evaluated first atom, and calculation using the weighted values.
